(19) United States
(12) Patent Application Publication
(10) Pub. No.: US 2024/0117317 A1
(43) Pub. Date: Apr. 11, 2024

(54) GENERATION OF INDUCED PLURIPOTENT STEM CELL LINES FROM HUMAN PATIENTS WITH MUTATIONS IN THE GLUCOKINASE GENE

(71) Applicant: Qatar Foundation for Education, Science and Community Development, Doha (QA)

(72) Inventors: Essam Abdelalim, Doha (QA); Khalid Hussain, Doha (QA)

(21) Appl. No.: 18/276,416

(22) PCT Filed: Feb. 7, 2022
(86) PCT No.: PCT/QA2022/050001

§ 371 Date: Aug. 8, 2023

Related U.S. Application Data
(60) Provisional application No. 63/146,930, filed on Feb. 8, 2021.

(51) Int. Cl. C12N 5/071; A61K 35/39; C12Q 1/6869

(52) U.S. Cl. CPC C12N 5/0676; A61K 35/39; C12Q 1/6869; C12Q 2600/156

(57) ABSTRACT

Heterozygous and homozygous mutations in the glucokinase (GCK) gene lead to maturity-onset diabetes of the young type 2 (MODY2) and permanent neonatal diabetes (PNDM), respectively. The present invention relates to a method for generating induced pluripotent stem cell (iPSC) lines from patients with MODY2 and PNDM due to mutations in the GCK gene. The generated iPSC lines are able to differentiate into the three germ layers and show normal karyotypes. These iPSC lines can serve as valuable human cell models for understanding diabetes pathogenesis and developing new therapies for diabetes.

### BACKGROUND

Diabetes mellitus (DM) is a metabolic disease characterized by chronic hyperglycemia resulting from progressive loss of pancreatic beta-cells, which could lead to several debilitating complications. Different paths, triggered by several genetic and environmental factors, lead to the loss of pancreatic beta-cells and their function. Understanding these many paths to beta-cell damage or dysfunction could help in identifying therapeutic approaches specific for each path.

Most of our knowledge about diabetes pathophysiology has been obtained from studies on animal models, which do not fully correspond with human diabetes phenotypes. Currently, human pluripotent stem cell (hPSC) technology is a powerful tool for generating in vitro human models, which could provide key information about the disease pathogenesis and provide cells for personalized therapies. Recent progress in somatic cell reprogramming has allowed the generation of induced pluripotent stem cells (iPSCs) from diabetic subjects. iPSCs have the capacity to differentiate into insulin-producing cells, which display key properties of beta-cells, including glucose-stimulated insulin secretion upon maturation in vivo.

Glucokinase (GCK) is a key regulatory enzyme in the pancreatic beta-cell. GCK plays a crucial role in regulating insulin secretion and has been termed the “pancreatic beta-cell sensor.” Given its vital role in insulin release regulation, it is understandable that mutations in the gene encoding GCK can cause hyperglycemia and hypoglycemia. Heterozygous mutations in the GCK gene can cause maturity-onset diabetes of the young (MODY), characterized by mild hyperglycemia, which is present at birth but is often only detected later in life during screening for other purposes. Homozygous mutations in the GCK gene lead to a more severe phenotype, presenting at birth as permanent neonatal diabetes mellitus (PNDM).

MODY accounts for 1 to 5 percent of all instances of diabetes in the United States, and MODY2, caused by mutations in the GCK gene, accounts for 8 percent to 60 percent of all MODY cases, depending on population sampling. GCK links blood glucose levels to insulin secretion by converting glucose to glucose-6-phosphate, the rate-limiting step in glycolysis. The catalytic capacity of GCK in beta-cells determines the threshold for glucose-stimulated insulin secretion.

### BRIEF DESCRIPTION OF THE DRAWINGS

FIG. 1 shows that Sanger sequencing analysis confirmed the GCK mutation (c.437 T>C) in the generated iPSC lines.

FIG. 2 shows that the iPSC lines, QBRIi010-A and QBRIi011-A, exhibited a typical morphology of human embryonic stem cells (hESCs).

FIG. 3 shows that the iPSC lines, QBRIi010-A and QBRIi011-A, expressed the key pluripotency markers, including OCT4, NANOG, SOX2, SSEA4, TRA-1-60, and TRA-1-81 as examined by immunocytochemistry.

FIG. 4 shows the expression of pluripotency markers confirmed by RT-PCR.

FIG. 5 shows the expression of pluripotency markers confirmed by qPCR.

FIG. 6 shows that QBRIi010-A and QBRIi011-A silenced the expression of exogenous Sendai viral vector after several passages as confirmed by RT-PCR at passage 22.

FIG. 7 shows that both cell lines were able to form embryoid bodies (EBs) upon spontaneous differentiation.

FIG. 8 shows that both cell lines expressed specific markers of the three germ layers, including NESTIN and NEUROD1 (ectoderm), brachyury (T) (mesoderm), and SOX17 (endoderm).

FIG. 9 shows that the generated cell lines passed the scorecard analysis with high scores for the three germ layers, and lost the pluripotency expression upon spontaneous differentiation.

FIG. 10 shows that karyotype analysis of both iPSC lines and the patient's blood samples showed normal karyotype with a cytogenetic balanced pericentric inversion within chromosome 9 (46,XY,inv(9) (p11q13)).

FIG. 11 shows that karyotype analysis of both iPSC lines and the patient's blood samples showed normal karyotype with a cytogenetic balanced pericentric inversion within chromosome 9 (46,XY,inv(9) (p11q13)).

FIG. 12 shows that RT-PCR analysis confirmed that these iPSC lines are not contaminated with mycoplasma.

FIG. 13 shows that RT-PCR analysis confirmed that these iPSC lines are not contaminated with mycoplasma.

### DETAILED DESCRIPTION

The present disclosure provides methods for generating induced pluripotent stem cell (iPSC) lines from patients with maturity-onset diabetes of the young type 2 (MODY2) and permanent neonatal diabetes (PNDM) due to mutations in the Glucokinase (GCK) gene. Disclosed iPSC lines can serve as human cell models for elucidating the underlying mechanism of GCK-associated diabetes and developing novel therapies for diabetes. The disclosed well-characterized iPSC lines that are generated from human patients with mutations in the GCK gene offer significant advantages over genetically manipulated animal models or human subjects for preclinical testing of therapeutic strategies and for drug screening as well as for studies designed to gain insight into the molecular mechanisms of diabetes due to mutations in the GCK gene.

In one aspect, the instant disclosure provides methods of producing iPSC lines from patients with MODY2 or PNDM. In embodiments, the methods comprise:
a. obtaining peripheral blood mononuclear cells (PBMCs) of patients with mutations in the GCK gene, for example wherein heterozygous mutations in the GCK gene cause MODY2, and homozygous mutations in the GCK gene cause PNDM;
b. identifying heterozygous or homozygous mutations in the GCK gene in the PBMCs, for example using whole exome sequencing (WES);
c. confirming the heterozygous or homozygous mutations in the GCK gene in the PBMCs, for example using Sanger sequencing;
d. reprogramming the PBMCs into the iPSC lines;
e. selecting and expanding the reprogrammed iPSC lines;
f. confirming the heterozygous or homozygous mutations in the GCK gene in the iPSC lines using, for example, Sanger sequencing; and
g. confirming the expression of pluripotency markers in the iPSC lines.

The disclosed methods can be used to establish iPSC lines for, for example, disease modeling. For example, iPSC lines from human patients with mutations in the GCK gene will carry the same genetic information as the patients. Therefore, iPSC lines can be used by many researchers to generate pancreatic islet cells and liver cells (hepatocytes) as well as other cells expressing GCK, to understand how GCK mutations lead to disease, particularly diabetes. In addition, in embodiments these iPSC lines can be used instead of using mouse models, which do not reflect human physiology.

In some embodiments, the iPSC lines described herein can be used for cellular therapy. For example, using CRISPR-Cas9 gene-editing technology, it is possible to correct the mutation in the GCK gene of iPSC lines and generate a genetically identical iPSC line without the mutation in the GCK gene. In embodiments, this corrected iPSC line can produce normal pancreatic beta-cells that can be used for transplantation therapy.

In some embodiments, iPSC lines have the potential to transform drug discovery by providing physiologically relevant human cells (beta-cells and hepatocytes) for compound identification, target validation, compound screening, and tool discovery. This allows potential drug compounds to be screened in high-throughput systems using human cells generated from iPSC lines. In addition, iPSC lines can be used for toxicology screening to assess the safety of compounds or drugs within living cells.

The following non-limiting Example is provided for illustrative purposes only in order to facilitate a more complete understanding of representative embodiments. This example should not be construed to limit any of the embodiments described in the present specification.

### Example 1

1—Resource Table

1. Resource Table

| | |
|---|---|
| Unique stem cell lines | QBRIi010-A QBRIi01 1-A |
| identifier | |
| Alternative name(s) of | GCK-MODY2 iPSCs (QBRIi010-A) |
| stem cell line | GCK-PNDM iPSCs (QBRIi011-A) |
| Institution | Qatar Biomedical research Institute (QBRI), |
| | Hamad Bin Khalifa University (HBKU), |
| | Qatar Foundation, Doha, Qatar |
| Contact information of | Essam M. Abdelalim |
| distributor | (emohamed@hbku.edu.qa) |
| Type of cell line | iPSC |
| Origin | human |
| Cell Source | Blood |
| Clonality | Clonal |
| Method of | Integration-free Sendai virus vector |
| reprogramming | contain OCT3/4, SOX2, c-MYC, and KLF4 |
| Genetic Modification | YES |
| Type of Modification | Hereditary |
| Associated disease | Patient 1: (Maturity diabetes of |
| | the young type 2 (MODY2) |
| | Patient 2: Permanent neonatal |
| | diabetes mellitus (PNDM) |
| Gene/locus | Gene: GCK |
| | Locus: 7p13 |
| | Heterozygous mutation: c.437 T > C |
| | in exon 4 (Patient 1) |
| | Homozygous mutation: c.437 T > C |
| | in exon 4 (Patient 2) |
| Method of modification | N/A |
| Name of transgene or | N/A |
| resistance | |
| Inducible/constitutive | N/A |
| system | |
| Date archived/ | Date cell line archived or deposited |
| stock date | in repository |
| Cell line | N/A |
| repository/bank | |
| Ethical approval | The protocol was approved by the |
| | Institutional Review Board (IRB) |
| | of Sidra Medicine (no. 1702007608) |
| | and QBRI (no. 2018-002) |

2—Resource Utility

Two iPSC lines were established from patients with MODY2 and PNDM due to heterozygous and homozygous mutations in the GCK gene (c.437 T>C), respectively. These iPSC lines will serve as human cell models for elucidating underlying mechanism of GCK-associated diabetes and developing novel therapies for diabetes.

3—Resource Details

Glucokinase (GCK) gene encodes an enzyme that phosphorylate glucose to glucose-6-phosphate during glycolysis. This is the rate limiting step in glucose metabolism and enables pancreatic p-cells and hepatocytes to respond appropriately to blood glucose level. Patients with GCK mutations have reduced glycolysis, altered intracellular ADP/ATP ratio that affect potassium channel and thus results in impaired insulin secretion. Heterozygous mutations in GCK gene has been reported to cause maturity onset diabetes of young type 2 (MODY2), while homozygous mutations in GCK leads to permanent neonatal diabetes mellitus (PNDM). Here, we generated two iPSC lines, QBRIi010-A and QBRIi011-A, from patients with MODY2 and PNDM, respectively. QBRIi010-A was generated from a 54-year-old male patient with MODY2 (patient 1) due to a heterozygous mutation (c.437 T>C, p.L146P) in the GCK gene.

TABLE 1
Table 1
Summary of lines.

| IPSC line | Abbreviation | | | | Genotype | |
|---|---|---|---|---|---|---|
| names | in figures | Gender | Age | Ethnicity | of locus | Disease |
| QBRIi010-A | QBRIi010-A | Male | 54 years | Egyptian | Heterozygous | Maturity-onset diabetes |
| | | | old | | (GCK, c.437 | of the young |
| | | | | | T > C p.L146P) | type 2 (MODY2) |
| QBRIi011-A | QBRIi011-A | Male | 11 years | Egyptian | Homozygous | Permanent neonatal |
| | | | old | | (GCK, c.437 | diabetes |
| | | | | | T > C p.L146P) | mellitus (PNDM) |

Furthermore, QBRIi011-A was generated from an 11-year-old male patient with PNDM (patient 2) due to a homozygous mutation (c.437 T>C, p.L146P) in the GCK gene (Table 1). Patient 2 was diagnosed with diabetes at one-day-old and was permanently on insulin treatment. The GCK mutations were identified in the patient's sample using whole exome sequencing (WES) and was further confirmed by Sanger sequencing.

The mutation (c.437 T>C) in the GCK gene leads to the substitution of leucine to proline at position 146 (p.L146P). For iPSC generation, the peripheral blood mononuclear cells (PBMCs) were isolated from patient's blood and transduced with non-integrating Sendai virus expressing OCT3/4, SOX2, c-MYC and KLF4 transcription factors.

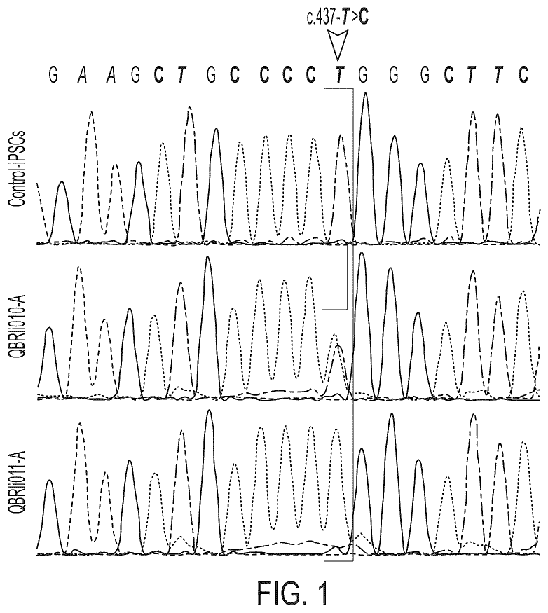

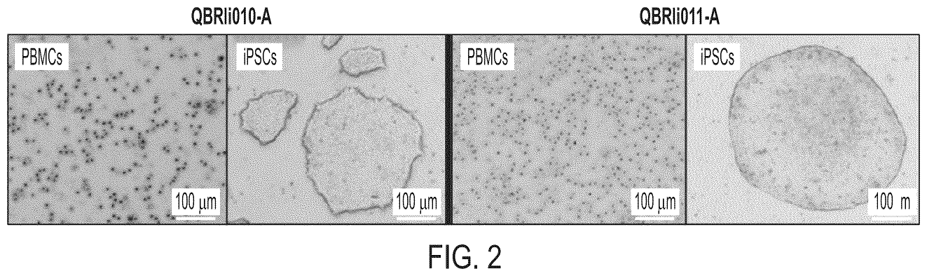

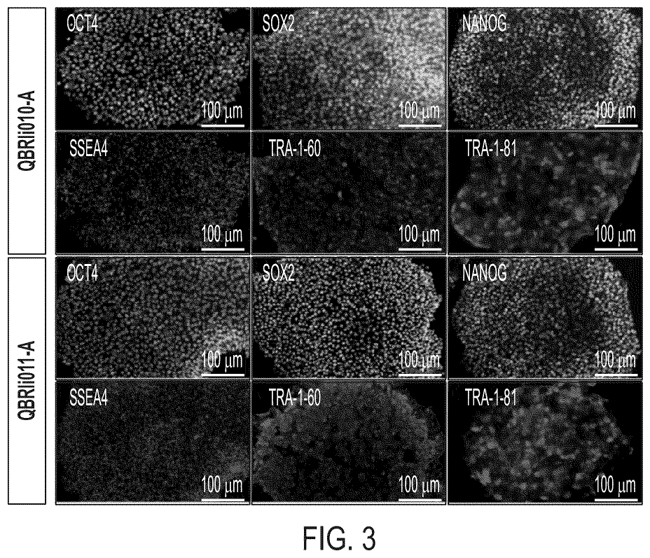

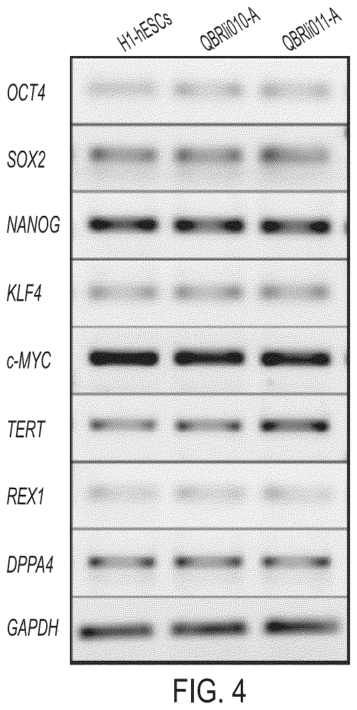

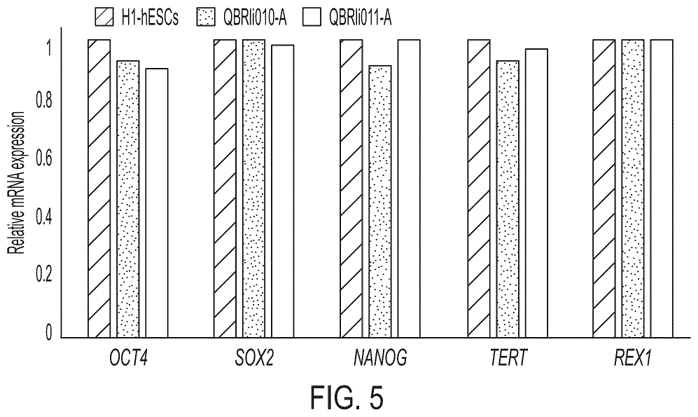

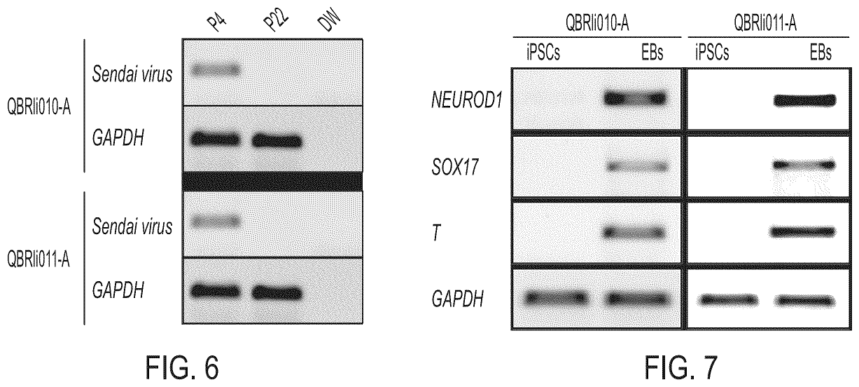

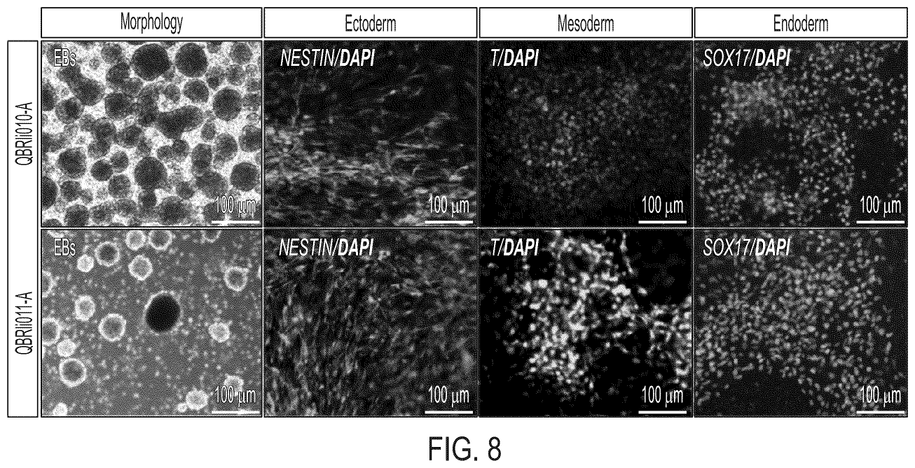

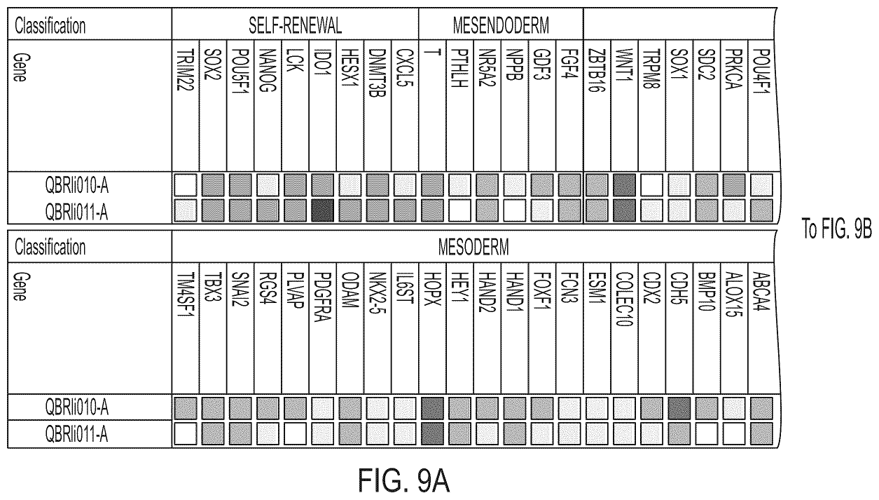

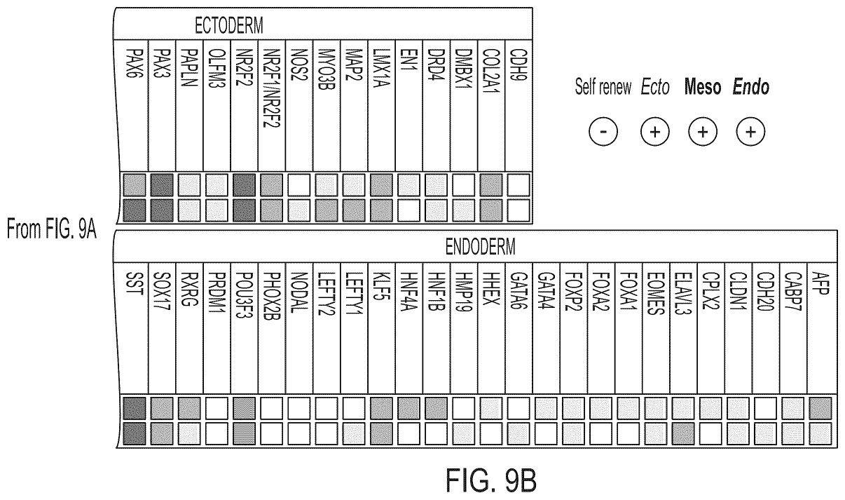

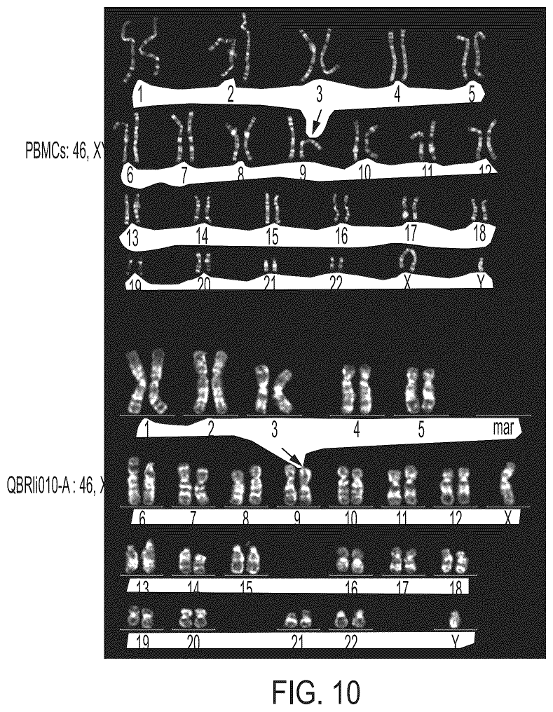

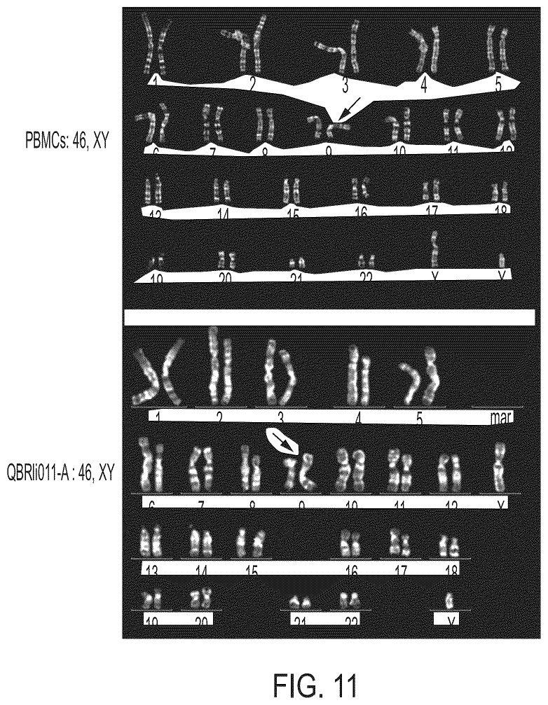

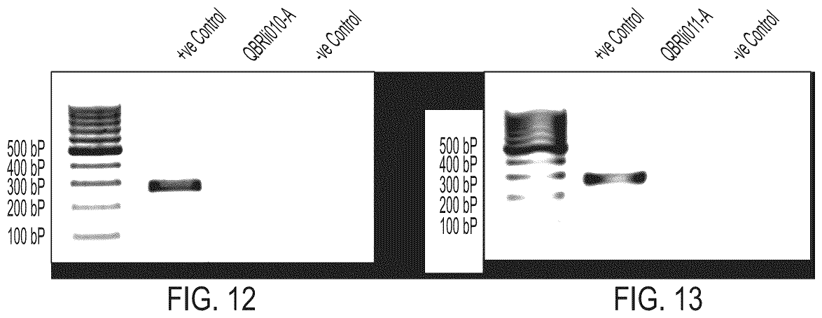

The generated iPSC-like colonies were picked and expanded for further characterization (Table 2; “Supplementary FIG. 1” refers to FIGS. 10-13). Sanger sequencing analysis confirmed the GCK mutation (c.437 T>C) in the generated iPSC lines (FIG. 1). The coding sequence used as a reference sequence is the NCBI sequence (NM_000162.4). The iPSC lines, QBRIi010-A and QBRIi011-A, exhibited a typical morphology of human embryonic stem cells (hESCs) (FIG. 2) and expressed the key pluripotency markers, including OCT4, NANOG, SOX2, SSEA4, TRA-1-60, and TRA-1-81 as examined by immunocytochemistry (FIG. 3). The expression of pluripotency markers were further confirmed by RT-PCR and qPCR (FIGS. 4, 5). QBRIi010-A and QBRIi011-A silenced the expression of exogenous Sendai viral vector after several passages as confirmed by RT-PCR at passage 22 (FIG. 6). Karyotype analysis of both iPSC lines and the patient's blood samples showed normal karyotype with a cytogenetic balanced pericentric inversion within chromosome 9 (46,XY,inv(9) (p11q13) (FIGS. 10-11), which is a normal variant with no clinical significance. Both cell lines were able to form embryoid bodies (EBs) upon spontaneous differentiation and expressed specific markers of the three germ layers, including NESTIN and NEUROD1 (ectoderm), brachury (T) (mesoderm), and SOX17 (endoderm) (FIGS. 7, 8). The generated cell lines passed the scorecard analysis with high scores for the three germ layers and lost the pluripotency expression upon spontaneous differentiation (FIG. 9). RT-PCR analysis confirmed that these iPSC lines are not contaminated with mycoplasma (FIGS. 12-13). The origin of the iPSC lines were confirmed by short tandem repeat (STR) profiling, which confirmed the same genetic identity of the patient's PBMCs.

4. Materials and Methods

4.1. Cell Culture and Reprogramming

Blood samples were collected from the donors with informed consent and PBMCs were isolated using Ficoll-Paque (Sigma-Aldrich). The cells were cultured in StemPro-34 complete medium (Gibco) supplemented with FLT3 (100 ng/ml), IL6 (20 ng/ml), TPO (100 ng/ml, SCF (100 ng/ml) for four days before reprogramming. The cells were reprogrammed using CytoTune-iPS 2.0 Sendai reprogramming kit (Thermo Fisher Scientific). Established iPSC clones were cultured onto plates coated with Geltrex and fed with StemFlex medium (ThermoFisher Scientific).

TABLE 2
Characterization and validation.

| Classification | Test | Result | Data |
|---|---|---|---|
| Morphology | Photography | Normal for both lines | FIG. 1 panel B |
| Phenotype | Qualitative analysis | Assess staining/expression of pluripotency markers: | FIG. 1 panel C, and D |
| | Immunocytochemistry | OCT4, SOX2, NANOG, SSEA4, TRA-1-60, | |
| | RT-PCR | TRA-81, TERT, [symbol] EXI, DPPA4, | |
| | | c-MYC, and [symbol] LP4 | |
| | Quantitative analysis | OCT4, SOX2, NANOG, TERT, [symbol] are positive | FIG. 1 panel [symbol] |
| | RT-RT-qPCR | for both cell lines and are similar to hESCs | |
| Genotype | Karyotype (G-banding) and | Both 46XY with mean resolution of 300 | Supplementary |
| | resolution | | FIG. [symbol] panels |
| | | | A, and B |
| Identify | Microsatellite PCR (mPCR) | N/A | N/A |
| | OR STR analysis | 15 loci, 100% matched | Submitted in archive |
| | | | with journal |
| Mutation | Sequencing | Heterozygous and homozygous | FIG. 1 panel A |
| analysis | | mutations (c.437 T > C) | |
| | Southern [symbol] ot | N/A | N/A |
| | OR WGS | | |
| Microbiology | Mycoplasma | Mycopl [symbol] testing by RT-PCR | Supplementary FIG. [symbol] |
| and virology | S [symbol] virus | Negative by RT-PCR | panels C, and D |
| | | | FIG. [symbol] panel [symbol] |
| Differ [symbol] | Embryoid body formation | The embryoid body forced and express NESTIN, | FIG. [symbol] panel G, H and I |
| potential | and Scorecard | BRACHYURY and SOX17 (immunostaining and | |
| | | RT-PCR) as well as the expression | |
| | | of N [symbol] ODI (RT-PCR) | |
| Donor screening | HIV 1 + 2 Hepatitis | N/A | N/A |
| (OPTIONAL) | B, Hepatitis C | | |
| Genotype additional | Blood group genotyping | N/A | N/A |
| info (OPTIONAL) | HLA [symbol] typing | N/A | N/A |

[symbol]

indicates data missing or illegible when filed

TABLE 3
Reagents details.
Antibodies used for immunocytochemistry

| | | | Company Car # |
|---|---|---|---|
| | Antibody | Dilution | and RRID |
| Pluripotency | Rabbit anti-OCT4 | 1:500 (IF) | Cell Signaling |
| Markers | | | Technology |
| | | | Cat#9656, |
| | | | RRID: AB_3668242 |
| Pluripotency | Rabbit anti-SOX2 | 1:500 (IF) | Cell Signaling |
| Markers | | | Technology |
| | | | Cst#9656, |
| | | | RRID: AB_1688242 |
| Pluripotency | Rabbit anti-NANOG | 1:500 (IF) | Cell Signaling |
| Markers | | | Technology |
| | | | Cat#9656, |
| | | | RRID: AB_3658242 |
| Pluripotency | Mouse anti SSEA4 | 1:500 (IF) | Cell Signaling |
| Markers | | | Tectumlogy |
| | | | Car#9656, |
| | | | RRID: AB_1658242 |
| Pluripotency | Mouse anti TRA-1-60 | 1:500 (IF) | Cell Signaling |
| Markers | | | Technology |
| | | | Cat#9656, |
| | | | RRID: AB_1658242 |
| Pluripotency | Mouse anti TRA-81 | 1:500 (IF) | Cell Signaling |
| Markers | | | Technology |
| | | | Cat#9656, |
| | | | RRID: AB_3658243 |
| Differentiation | Mouse Anti-Human | 1:500 | R and D Systems |
| Markers | Nestin Monoclonal | | Cat#MAB1259, |
| | antibody | | RRID: AB_2251384 |
| Differentiation | Sox17 mouse | 1:2000 | O [symbol] iGene |
| Markers | monoclonal antibody, | | Cat#TA500096, |
| | clone 208 | | RRID: AB_2255344 |
| Differentiation | Mouse anti- | | Abcam Cat#ab140661 |
| Markers | Brachyury | | |
| Secondary | Donkey anti-rabbit | 1:500 | Thermo Fisher Scientific |
| antibodies | IgG (H + L) highly | | Cat#A-21206, |
| | Cross-adsorbed | | RRID: AB_2535792 |
| | secondary Antibody, | | |
| | Alexa Fluor 488 | | |
| Secondary | Donkey anti-mouse | 1:500 | Thermo Fisher Scientific |
| antibodies | IgG (H + L) highly | | Cat#A10037. |
| | Cross-adsorbed | | RRID: AB_2524013 |
| | secondary Antibody, | | |
| | Alexa Fluor 568 | | |

| Primers | Target | Forward/Reverse primer (5′-3′) |
|---|---|---|
| Pluripotency | OCT4 | GACAGGGGGAGGGGAGGAGCT AGG/ |
| Markers | | CTTCCCTCCAACCAGTTGCCCCA AAC |
| (RT-PCR/RT-qPCR) | | |
| Pluripotency | SOX2 | GGGAAATGGGAGGGGTGCAAA AGAGG/ |
| Markers | | TTGCGTGAGTGTGGATGGGATGGGATTG |
| (RT-PCR/RT-qPCR) | | GTG |
| Pluripotency | c-MYC | GCGTCCTGGGAAGGGAGATCCG GAGC/ |
| Markers | | TTGAGGGGCATCGTCGCGGGAG |
| (RT-PCR) | | GCTG |
| Pluripotency | KLP4 | CCCAA TTACCCATCCTTCCT/ |
| Markers | | ACGATCGTCTTCCCCTCTTT |
| (RT-PCR) | | |
| Pluripotency | NANOG | CATGA GTGTGGATCCAGCTTG/ |
| Markers | | CCTGAATAAGCAGATCCATGG |
| (RT-PCR/RT-qPCR) | | |
| Pluripotency | REX1 | TGACAG TCCAGCAGGTGTTTG/ |
| Markers | | TCTTGTCTTTGCCCGTTTCT |
| (RT-PCR/RT-qPCR) | | |
| Pluripotency | TERT | CCTGCTCAAGCTGACTCGACACCGTG/ |
| Markers | | GGAAAAGCTGGCCCTGGGGTGGAGC |
| (RT-PCR/RT-qPCR) | | |
| Pluripotency | DPPA4 | GGAGCCGCCTGCCCTGGAAAATTC/ |
| Markers | | TTTTTCCTGATATTCTATTCCCAT |
| (RT-PCR) | | |
| Sendi | SENDAI VIRUS | GGATCACTACCTGATATCGAGC/ |
| virus | | ACCAGACAAGAGTTTAAGAGATATGTATC |
| Ectodermal | NEUROD1 | CGAATTTGGTGTGGCTGTATTC/ |
| differentiation | | GGAGAGGAAAGAAGTGGTAAGG |
| Mesodermal | BRACHYURY | GCCCTCTCCCTCOCCTCCACGCACAG/ |
| differentiation | | CGGCGCCGTTGCTCACAGACCACAGG |
| Endodermal | SOX17 | TCCTGGAGGAGCTAAGGAAA/ |
| differentiation | | GCCACTTCCCAAGGTGTAAA |
| House-Keeping | GAPDH | AGGACCACTTTGTCAAGCTCATTTC/ |
| Genes | | GCAGTGAGGGTCTCTCTCTTCTGT |
| (RT-PCR) | | |
| Targeted | GCK | GATCTCCCTTCTGAGCACATG/ |
| mutation | | TCCCTGACCAATAGCTTGGCTTG |
| anolysis/ | | |
| sequencing | | |
| Mycoplasma | Mycoplasma | CGGAGCAAACAGGATTAGATACCCT/ |
| primer | | TGCACCATCTGTCACTCTGTTAACCTC |

[symbol]

indicates data missing or illegible when filed

4.2. Immunocytochemistry

Cells were fixed with 4% paraformaldehyde in 0.1 M PBS for 20 min, permeabilized with 0.5% Triton X-100 (Sigma-Aldrich) in 0.1 M PBS and blocked with 6% bovine serum albumin. The cells were incubated with primary antibodies at 4° C. overnight (Table 3), then washed with 0.3% Tween-20 in 0.1 M PBS and incubated with the secondary antibodies (Table 3) for 1 h at room temperature. Images were acquired using an inverted fluorescence microscope (Olympus IX 53).

4.3. Sanger Sequencing

Genomic DNA was extracted using quick extract genomic DNA extraction buffer (epicenter). The region of GCK spanning the mutation was amplified using PCR-Master mix (ThermoFisher Scientific) and specific primers (Table 3). The PCR products were purified and sequenced.

4.4. Karyotype Analysis

The cells were processed using standard protocols for G-banding. Briefly, to arrest the cells at the metaphase, they were treated with 100 ng/ml KaryoMax colcemid (ThermoFisher Scientific). The arrested cells were further exposed to 0.75 M KCL hypotonic solution (ThermoFisher Scientific) for 20 min at 37° C. and then fixed with methanol: glacial acitic acid (3:1). 20 metaphases were karyotyped for each sample.

4.5. Gene Expression Analysis

Total RNA was isolated using direct-zol RNA MiniPrep kit (Zymo Research) according to the manufacturer's instructions and complementary DNA was synthesized using SuperScript IV First-Strand Synthesis System (Thermo Fisher Scientific). quantitative PCR (qPCR) was performed using GoTaq qPCR Master (Promega) with the primers listed in Table 3, using H1-hESCs as a positive control and gene expression was normalized to GAPDH.

4.6. Embryoid Body (EB) Formation and Scorecard Analysis

iPSCs were detached as small clumps and plated in ultra-low attachment plates in DMEM/F12 medium supplemented with 20% Knockout Serum Replacement, 1 mM L-glutamine, 1% non-essential aminoacids, 0.1 mM 2-beta-mercaptoethanol, 1% (v/v) penicillin—streptomycin for 4 days. EBs were then plated on geltrex coated plates for 14 days and examined for the expression of all germ layers markers using RT-PCR and immunostaining. Scorecard analysis was performed using the TaqMan hPSC Scorecard assay (Life Technologies, A15876).

TaqMan master mix was added to the diluted cDNA. 10 μl was loaded per well into hPSC Scorecard plate and run on a QuantStudio7 Flex Real-Time PCR system (Applied Biosystems). The results were analysed using an online TaqMan hPSC Scorecard analysis software (https://www.thermofisher.com/qa/en/home/life-science/stem-cell-research/taqman-hpsc-scorecard-panel/scorecard-software. html).

4.7. Short Tandem Repeat Profiling (STR)

STR was performed using AmpFISTR Identifiler Plus PCR amplification Kit (Applied biosynthesis, Life Technologies) according to the manufacturer's instructions.

4.8. Mycoplasma Detection Test

The cells were regularly checked for the absence of mycoplasma contamination in the culture media using PCR with the primers listed in Table 3.

SUPPLEMENTARY TABLE 1
Short tandem repeat (STR) analysis of iPSC line (QBRIi010-A) generated
from a patient with MODY2 due to a heterozygous mutation in the
GCK gene. STR analysis authenticated the identity of the cell
line with the parental PBMCs using 15 different loci.

| | |
|---|---|
| Chromosome | Alleles |

| Locus | location | PBMCs | QBRIi010-A |
|---|---|---|---|
| D8S1179 | 8 | 13, 16 | 13, 16 |
| D21S11 | 22q11.2-q21 | 29, 30 | 29, 30 |
| D7S820 | 7q11.21-22 | 8, 9 | 8, 9 |
| CSF1PO | 5q33.3-34 | 11, 12 | 11, 12 |
| D3S1358 | 3p | 18 | 18 |
| TH01 | 11p15.5 | 7, 9.3 | 7, 9.3 |
| D13S317 | 13q22-31 | 10, 13 | 10, 13 |
| D16S539 | 16q24-qter | 8, 11 | 8, 11 |
| D2S1338 | 2q35-37.1 | 17, 24 | 17, 24 |
| AMEL | X: p22.1-22.3. | X, Y | X, Y |
| | Y: p11.2 | | |
| D5S818 | 5q21-31 | 12, 13 | 12, 13 |
| FGA | 4q28 | 19, 22 | 19, 22 |
| D19S433 | 19q12-13.1 | 15, 16 | 15, 16 |
| vWA | 12p12-pter | 17, 19 | 17, 19 |
| TPOX | 2p23-2per | 8 | 8 |
| D18S51 | 18q21.3 | 15, 20 | 15, 20 |

Without further elaboration, it is believed that one skilled in the art can use the preceding description to utilize the claimed inventions to their fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles discussed. In other words, various modifications and improvements of the embodiments specifically disclosed in the description above are within the scope of the appended claims. For example, any suitable combination of features of the various embodiments described is contemplated.

1-17. (canceled)

18. A method for generation of induced pluripotent stem cell (iPSC) lines from patients with mutations in a gene encoding glucokinase (GCK), the method comprising:
a) obtaining peripheral blood mononuclear cells (PBMCs) of patients with mutations in the GCK gene, wherein heterozygous mutations in the GCK gene cause maturity-onset diabetes of the young type 2 (MODY2), and homozygous mutations in the GCK gene cause permanent neonatal diabetes mellitus (PNDM);
b) identifying heterozygous or homozygous mutations in the GCK gene in the PBMCs using whole exome sequencing (WES);
c) confirming the heterozygous or homozygous mutations in the GCK gene in the PBMCs using Sanger sequencing;
d) reprogramming the PBMCs into the iPSC lines;
e) selecting and expanding the reprogrammed iPSC lines;
f) confirming the heterozygous or homozygous mutations in the GCK gene in the iPSC lines using Sanger sequencing; and
g) confirming the expression of pluripotency markers in the iPSC lines.

19. The method according to claim 1, further comprising using the iPSC lines to produce normal pancreatic beta-cells.

20. The method according to claim 2, wherein the normal pancreatic beta-cells are used in transplantation therapy.

21. The method according to claim 1, wherein the pluripotency markers comprise at least one of OCT4, NANOG, SOX2, TRA-1-60, TRA81, and SSEA4.

22. The method according to claim 4, wherein the pluripotency markers comprise OCT4.

23. The method according to claim 4, wherein the pluripotency markers comprise NANOG.

24. The method according to claim 4, wherein the pluripotency markers comprise SOX2.

25. The method according to claim 4, wherein the pluripotency markers comprise TRA-1-60.

26. The method according to claim 4, wherein the pluripotency markers comprise TRA81.

27. The method according to claim 4, wherein the pluripotency markers comprise SSEA4.

28. The method according to claim 1, wherein the iPSC lines form embryoid bodies (EBs) upon spontaneous differentiation and express specific markers of the three germ layers, including NESTIN and NEUROD1 (ectoderm), brachyury (T) (mesoderm), and SOX17 (endoderm).

29. Induced pluripotent stem cells (iPSC) from patients with mutations in a gene encoding glucokinase (GCK), made by a method comprising:
a) obtaining peripheral blood mononuclear cells (PBMCs) of patients with mutations in the GCK gene, wherein heterozygous mutations in the GCK gene cause maturity-onset diabetes of the young type 2 (MODY2), and homozygous mutations in the GCK gene cause permanent neonatal diabetes mellitus (PNDM);
b) identifying heterozygous or homozygous mutations in the GCK gene in the PBMCs using whole exome sequencing (WES);
c) confirming the heterozygous or homozygous mutations in the GCK gene in the PBMCs using Sanger sequencing;
d) reprogramming the PBMCs into the iPSC lines;
e) selecting and expanding the reprogrammed iPSC lines;
f) confirming the heterozygous or homozygous mutations in the GCK gene in the iPSC lines using Sanger sequencing; and
g) confirming the expression of pluripotency markers in the iPSC lines.

30. The induced pluripotent stem cells according to claim 12, wherein the method further comprises using the iPSC lines to produce normal pancreatic beta-cells, and wherein the normal pancreatic beta-cells are used in transplantation therapy.

31. The induced pluripotent stem cells according to claim 12, wherein the pluripotency markers comprise at least one of OCT4, NANOG, SOX2, TRA-1-60, TRA81, and SSEA4.

32. The induced pluripotent stem cells according to claim 14, wherein the pluripotency markers comprise OCT4.

33. The induced pluripotent stem cells according to claim 14, wherein the pluripotency markers comprise NANOG.

34. The induced pluripotent stem cells according to claim 14, wherein the pluripotency markers comprise SOX2.

35. The induced pluripotent stem cells according to claim 14, wherein the pluripotency markers comprise TRA-1-60.

36. The induced pluripotent stem cells according to claim 14, wherein the pluripotency markers comprise TRA81.

37. The induced pluripotent stem cells according to claim 14, wherein the pluripotency markers comprise SSEA4.
